(19) United States
(12) Patent Application Publication
(10) Pub. No.: US 2024/0161911 A1
(43) Pub. Date: May 16, 2024

(54) ARTIFICIAL INTELLIGENCE-BASED SCHEDULING OF MEDICAL PROCEDURES IN OPERATING ROOMS

(71) Applicant: OPERAIT HEALTH INC., Glendale, CA (US)

(72) Inventors: Budhaditya Syamal Chattopadhyay, Glendale, CA (US); Luis Felipe Gonzalez-Silen, New York, NY (US); Brian Lindley Merrell, Arlington, VA (US); Mark Dennis Schlesinger, New York, NY (US)

(21) Appl. No.: 18/389,228

(22) Filed: Nov. 14, 2023

Related U.S. Application Data
(60) Provisional application No. 63/425,463, filed on Nov. 15, 2022.

(51) Int. Cl. G16H 40/20; G06N 5/022

(52) U.S. Cl. CPC G16H 40/20; G06N 5/022

(57) ABSTRACT

An example method may include predicting one or more time slots for performing medical procedures in a hospital for a pre-determined time period by applying a trained artificial intelligence model on real-time operating room scheduling data associated with the hospital. One of the predicted time slots for performing a medical procedure on a patient in the hospital is allocated in response to a request for scheduling the medical procedure on the patient. The medical procedure is scheduled in one of a plurality of operating rooms associated with the hospital based on the allocated time slot when the operating room becomes available for performing the medical procedure.

### RELATED APPLICATIONS

Benefit is claimed under 35 U.S.C 119(e) to U.S. Provisional Patent Application Ser. No. 63/425,463 entitled “ARTIFICIAL INTELLIGENCE-BASED OPERATING ROOM SCHEDULING”, filed on Nov. 15, 2022”, by OPERAIT HEALTH INC., which is herein incorporated in its entirety by reference for all purposes.

### TECHNICAL FIELD

The present disclosure relates to healthcare management systems, and more particularly to methods, techniques, and systems for scheduling of medical procedures in operating rooms of hospitals using a trained artificial intelligence model.

### BACKGROUND

Typically, in hospitals, medical procedures such as surgical medical procedures are scheduled in operating rooms based on block times. Block time is when a specific doctor (or a surgical group) is manually assigned an operating room for a partial day or full day in advance. In such cases, the operating room belongs to a doctor or specific department for that block of time. The doctors can schedule medical procedures in the allocated operating room into that block of time. Thus, scheduling of medical procedures in a hospital may require co-ordination between hospital staff such as surgeons, anesthesiologists, nurses, technicians, and other support staff. Upon scheduling the medical procedure, tests and/or processes associated with the medical procedure are carried out on the patient. In other words, no tests and/or processes can be carried out on the patient till the medical procedure is scheduled in the operating room. Operating room schedules are usually published before the start of the day or at the end of the prior day. Normally, hospital staff refer to the published operating room schedule to determine which operating rooms they should be in, at what time, and for what type of medical procedure.

Often, on a given day in the future, operating rooms in a hospital appear as fully allocated due to the block times corresponding to these operating rooms owned by doctors or departments. This may even be the case when no medical procedures are actually scheduled in one or more of these operating rooms despite fully staffed operating rooms. Typically, doctors do not fully utilize their block times and release them very late. This leads to operating rooms not being utilized to their full capacity.

### BRIEF DESCRIPTION OF DRAWINGS

The drawings described herein are for illustrative purposes and are not intended to limit the scope of the present subject matter in any way:

FIG. 1 is a block diagram of an example cloud computing environment depicting a healthcare management system for scheduling medical procedures in operating rooms of respective hospitals;

FIG. 2 is a flow diagram illustrating an example method of scheduling a medical procedure on a patient in an operating room of a hospital;

FIG. 3 is a flow diagram illustrating an example method of predicting availability of one or more time slots for performing medical procedures in a hospital;

FIG. 4 is a flow diagram illustrating an example method of scheduling a medical procedure on a patient in a hospital based on a request for scheduling the medical procedure;

FIG. 5 is a flow diagram illustrating an example method of training artificial intelligence models to predict availability of time slots for performing medical procedures in a hospital;

FIG. 6 is a block diagram of an example healthcare management system including non-transitory computer-readable storage medium storing instructions to schedule a medical procedure in an operating room of a hospital using a trained artificial intelligence model;

FIG. 7 is a block diagram of an example healthcare management system for scheduling a medical procedure in an operating room of a hospital; and

FIG. 8 is a diagrammatic representation of a graphical user interface displaying predicted availability of a time slot in a virtual operating room.

### DESCRIPTION

Examples described herein may provide an enhanced computer-based and/or network-based method, technique, and system for scheduling medical procedures in operating rooms of hospitals using trained artificial intelligence model, Various embodiments are described with reference to the drawings, wherein like reference numerals are used to refer the drawings, wherein like reference numerals are used to refer to like elements throughout. In the following description, for the purpose of explanation, numerous specific details are set forth in order to provide thorough understanding of one or more embodiments. It may be evident that such embodiments may be practiced without these specific details.

In the present disclosure, a healthcare management system may predict availability of time slots (e.g., gaps in the operating room schedule) for performing a medical procedure in a hospital using a trained artificial intelligence model when physical operating rooms appear as fully occupied due to block times. The healthcare management system may allocate one of the predicted time slots for performing the medical procedure in the hospital so that necessary tests and/or processes are carried out on a patient undergoing the medical procedure. The healthcare management system may determine which of the operating rooms in the hospital are available based on the predicted time slots and schedule the medical procedure in one of the available operating rooms. Hence, additional requests for medical procedures can be accommodated using prediction of available time slots, leading to enhanced utilization of the operating rooms in the hospital. Advantageously, the healthcare management system may provide seamless patient experience, optimized staffing, and better utilization of hospital resources.

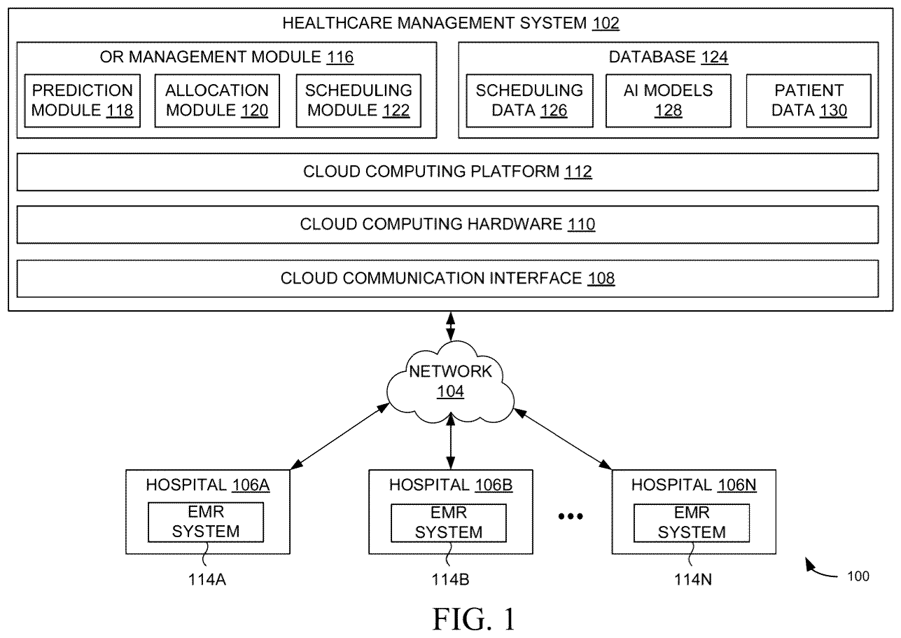

Referring now to the figures, FIG. 1 is a block diagram of an example cloud computing environment 100 depicting a healthcare management system 102 for scheduling medical procedures in operating rooms of respective hospitals. Example cloud computing environment 100 includes healthcare management system 102 communicatively coupled to Electronic Medical Record (EMR) systems 114A-114N of respective hospitals 106A-106N via a network 104. For example, network 104 can be a managed Internet protocol (IP) network administered by a cloud infrastructure service provider. In one example, network 104 may be implemented using wireless protocols and technologies, such as Wi-Fi, WiMAX, and the like. In other examples, network 104 can also be a packet-switched network such as a local area network, wide area network, metropolitan area network, Internet network, or other similar type of network environment. In yet other examples, network 104 may be a fixed wireless network, a wireless local area network (LAN), a wireless wide area network (WAN), a personal area network (PAN), a virtual private network (VPN), intranet or other suitable network system and includes equipment for receiving and transmitting signals. Each of hospitals 106A-106N may include operating rooms for performing medical procedures on patients, laboratories for conducting diagnostic tests on patients, and other facilities for diagnosis and treatment of patients. Hospitals 106A-106N may include electronic medical record (EMR) systems 114A-114N for managing schedule of operating rooms in hospitals 106A-106N.

Healthcare management system 102 may be deployed in a public, private or hybrid cloud computing environment. Healthcare management system 102 may enable doctors and hospital staff to schedule medical procedures in operating rooms of respective hospitals 106A-106N. Medical procedures may include any procedures which are carried out in operating rooms including surgical medical procedures (e.g., invasive and non-invasive) and non-surgical medical procedures. Operating room may include a unit in a hospital where the medical procedures are carried out such as operating theatre, procedure room, and the like. In some examples, healthcare management system 102 may predict availability of time slots for performing medical procedures in a hospital 106A. In these examples, healthcare management system 102 may apply a trained artificial intelligence model on real-time operating room scheduling data (e.g., scheduling data 126) to predict availability of time slots for a specific time period (i.e., weeks, month, or the like) when an EMR system 114A of hospital 106A indicates unavailability of operating rooms in hospital 106A during the specific time period.

When a request for scheduling a medical procedure in hospital 106A is received, for instance, from a doctor, healthcare management system 102 allocates one of the predicted time slots with a probability that at least one of the operating rooms in hospital 106A may become available prior to a particular time period (e.g., a day) of performing the medical procedure. This enables hospital 106A to carry out any tests and/or processes associated with the medical procedure on the patient. Until the day of the medical procedure, healthcare management system 102 may determine whether any operating room is available in hospital 106A for conducting the medical procedure on the patient. If healthcare management system 102 determines that an operating room has become available in hospital 106A, then healthcare management system 102 schedules the medical procedure in the available operating room in hospital 106A. Accordingly, EMR 114A of hospital 106A is updated with details associated with the medical procedure scheduled in the operating room.

As shown in FIG. 1, healthcare management system 102 includes a cloud communication interface 108, a cloud computing hardware 110, and a cloud computing platform 112. Cloud communication interface 108 may enable communication between cloud computing platform 112 and EMR systems 114A-114N via network 104. Cloud computing hardware 110 may include one or more servers on which an operating system is installed and including one or more processors, one or more storage devices for storing data, and other peripherals required for providing cloud computing functionality, Cloud computing platform 112 may be a platform which is capable of delivering functionalities such as data storage, data analysis, data visualization, data communication using cloud computing hardware 110 via application programming interfaces (APIs) and algorithms, and capable of delivering the aforementioned cloud services.

In the context of the present disclosure, cloud computing platform 112 employs an OR management module 116 and a database 124. OR management module 116 may be stored in the form of computer-readable instructions executable by cloud computing platform 112. OR management module 116 includes a prediction module 118, an allocation module 120, and a scheduling module 122. Prediction module 118 may create a virtual operating room for each of hospitals 106A-106N, In an example, a virtual operating room is a placeholder for additional requests for medical procedures especially when physical operating rooms in a hospital are blocked for other medical procedures as per EMR system of the hospital. For example, each virtual operating room may display predicted available time slots per working day in a calendar month. The virtual operating room may be of 8-hour duration. Prediction module 118 may predict availability of one or more time slats for accommodating additional requests of medical procedures in each of hospitals 106A-106N based on real-time scheduling data of hospitals 106A-106N. In one example, prediction module 118 may predict availability of time slot(s) associated with a hospital (e.g., the hospital 106A) by applying a trained artificial intelligence model on real-time operating room scheduling data 126. In this example, prediction module 118 may predict availability of time slots associated with physical operating rooms of hospital 106A for scheduling additional medical procedures for a specified time period (e.g., next 7 days, 14 days, 21 days, 30 days, or the like). Further, prediction module 118 may present the predicted time slots in the virtual operating room.

Furthermore, allocation module 120 may allocate one of the predicted time slots in the virtual operating room for performing a medical procedure on a patient in hospital 106A. In an example, allocation module 120 may allocate one of the predicted time slots in a virtual operating room associated with hospital 106A in response to a request for scheduling a medical procedure on a patient from a doctor/staff associated with hospital 106A, In one example, allocation module 120 allocates a predicted time slot in a virtual operating room based on a requested time duration on a specific date if available. In other example, allocation module 120 allocates a predicted time slot in a virtual operating room based on a requested time duration on any available date which is closer to the date specified in the request. When a predicted time slot (i.e., date, time, and duration) is allocated for a medical procedure to be performed on a patient, the medical procedure is considered as provisionally confirmed by hospital 106A. Advantageously, tests and/or processes related to the medical procedure on the patient may be initiated and completed by hospital 106A prior to the medical procedure once the time slot in the virtual operating room is allocated.

Scheduling module 122 may determine availability of a physical operating room in hospital 106A for the requested time duration for performing the medical procedure on the patient. Scheduling module 122 may schedule the medical procedure in the available operating room of hospital 106A for the requested time duration. Scheduling module 122 may update the virtual operating room details of the operating room against the allocated time slot. Also, scheduling module 122 may update EMR 114A of hospital 106A with details associated with the medical procedure on the patient. This may help doctors and hospital staff to prepare the scheduled operating room for the medical procedure and perform the medical procedure on the patient. In this manner, operating rooms in hospitals 106A-106N may be utilized efficiently.

Database 124 may store scheduling data 126, AI models 128, and patient data 130. Scheduling data 126 may include real-time operating room scheduling data associated with hospitals 106A-106N. For example, real-time operating room scheduling data 126 is received from EMR systems 114A-114N associated with hospitals 106A-106N. Real-time operating room scheduling data 126 may include schedule of medical procedures in operating rooms of each of hospitals 106A-106N. In an example, scheduling data 126 may be obtained from EMR systems 114A-114N via File transfer protocol upload/download (e.g., using SFTP). In another example, scheduling data 126 may be pushed directly by EMR systems 114A-114N to database 124 (e.g., BigQuery Table). In yet another example, scheduling data 126 may be obtained using Health Level Seven International (HL7) feed, wherein HL7 scheduling messages are placed in a queue, processed into a scheduling view, and stored in BigQuery table. AI models 128 may include trained artificial intelligence models for predicting one or more available time slots for performing medical procedures in each of hospitals 106A-106N using the real-time operating room scheduling data 126. Patient data 130 may include data associated with patients on which medical procedures are to be performed. Patient data 130 may include patient identifier, patient type, procedure identifier indicating a type of the medical procedure, doctors name, and the like. Patient data 130 may be used for allocating a time slot for performing a medical procedure and for scheduling the medical procedure in an operating room of a hospital. Healthcare management system 102 stores minimal Protected Health Information (PHI) of the patients such patient identifier and patient type.

Further, cloud computing environment 100 illustrated in FIG. 1 is shown purely for purposes of illustration and is not intended to be in any way inclusive or limiting to the embodiments that are described herein. For example, a typical cloud computing environment would include many more remote servers (e.g., physical host computing systems), which may be distributed over multiple data centers, which might include many other types of devices, such as switches, power supplies, cooling systems, environmental controls, and the like, which are not illustrated herein. It will be apparent to one of ordinary skill in the art that the example shown in FIG. 1, as well as all other figures in this disclosure have been simplified for ease of understanding and are not intended to be exhaustive or limiting to the scope of the idea.

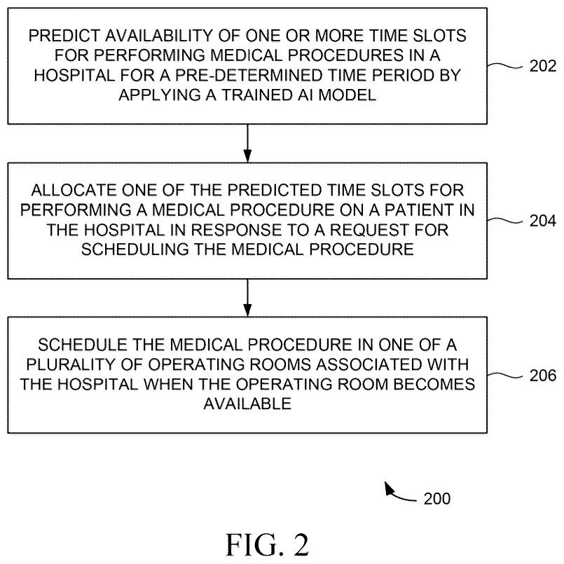

FIG. 2 is a flow diagram illustrating an example method 200 of scheduling a medical procedure on a patient in an operating room of a hospital. Example method 200 depicted in FIG. 2 represents generalized illustrations, and other processes may be added, or existing processes may be removed, modified, or rearranged without departing from the scope and spirit of the present application. In addition, method 200 may represent instructions stored on a computer-readable storage medium that, when executed, may cause a processor to respond, to perform actions, to change states, and/or to make decisions. Alternatively, method 200 may represent functions and/or actions performed by functionally equivalent circuits like analog circuits, digital signal processing circuits, application specific integrated circuits (ASICs), or other hardware components. Furthermore, the flow diagram is not intended to limit the implementation of the present application, but the flow diagram illustrates functional information to design/fabricate circuits, generate computer-readable instructions, or use a combination of hardware and computer-readable instructions to perform the illustrated processes.

At 202, availability of one or more time slots for performing medical procedures in the hospital for a specified time period may be predicted by applying a trained artificial intelligence (AI) model on real-time operating room scheduling data. For example, the real-time operating room scheduling data may include date of medical procedure, hospital name, operating room name, doctor's name, procedure name, scheduled procedure start time, scheduled time duration, and so on. In some examples, real-time operating room scheduling data may be pre-processed prior to feeding to a trained artificial intelligence model. In these examples, real-time operating room scheduling data may be aligned with a ground truth structure associated with a training data set. A binary value may be assigned to each minute of a day in real-time operating scheduling data across all operating rooms in a hospital. Also, ground truth minute representations may be augmented with various feature vectors extracted from real-time operating room scheduling data Feature vectors extracted from real-time scheduling data may provide context on which the trained artificial intelligence model can assess weighted predictive availability of time slots.

For example, it is determined whether one or more time slots for performing medical procedures in operating rooms are available in an Electronic Medical Record (EMR) system of the hospital. If it is determined that there is no availability of one or more time slots, the trained artificial intelligence model is applied on the real-time operating room scheduling data to predict availability of one or more time slots for performing medical procedures in the hospital. In one example, a trained artificial intelligence model may be binary supervised classification model such as linear regression model, support vector machine model, random forest model, and the like. In this example, real-time operating room scheduling data in a BigQuery table is fed to the trained artificial intelligence model. The trained artificial intelligence model may predict availability of time slots for performing additional medical procedures in the hospital. In some examples, a single actionable report containing available time slots per day for the specified time period may be generated.

At 204, one of the predicted time slots may be allocated for performing the medical procedure on the patient in the hospital. In an example, one of the available time slots in a virtual operating room is allocated in response to a request for scheduling the medical procedure from a doctor associated with the hospital. In this example, the time slot available in the virtual operating room is allocated based on date and time duration specified in the request. Based on the time slot allocated, necessary tests and/or processes are carried out on the patient.

At 206, the medical procedure may be scheduled in one of a plurality of operating rooms associated with the hospital based on the allocated time slot when the operating room becomes available for performing the medical procedure. For example, a blocker corresponding to the medical procedure on the patient is created in an EMR system of the hospital. The blocker may enable a staff of the hospital to prepare the operating room for performing the medical procedure on the patient on time. Further, a doctor or a surgeon may also visit the operating room according to the blocker in the EMR system to perform the medical procedure on the patient.

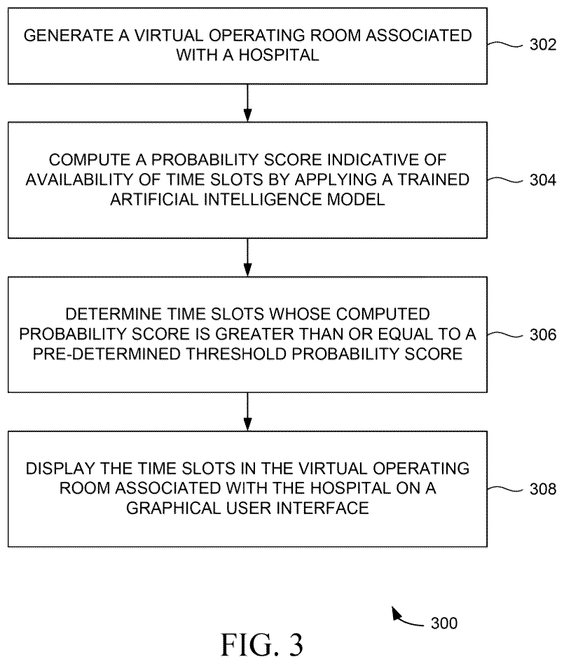

FIG. 3 is a flow diagram illustrating an example method 300 of predicting availability of one or more time slots for performing medical procedures in a hospital. At 302, a virtual operating room corresponding to a plurality of physical operating rooms in a hospital may be generated. For example, a virtual operating room may be a placeholder for additional medical procedures which are likely to be accommodated on a specific day when all physical operating rooms of a hospital appear unavailable, The virtual operating room may enable a temporarily block a time slot for a medical procedure so that necessary tests and/or processes can be initiated on a patient prior to the date of the medical procedure.

At 304, a probability score indicative of availability of each of the time slots for performing medical procedures in the hospital may be computed by applying a trained artificial intelligence model on the real-time operating room scheduling data of the hospital. In an example, the probability score may be computed on a minute-by-minute basis for each day of a specific number of days (e.g., next 30 days). The trained artificial intelligence model may assign a higher score to a minute availability if there is higher chance of one of operating rooms becoming available for that minute on a specific day and assign a lower probability score to a minute if there is lower possibility of one of operating rooms becoming available for that minute on that specific day. Given a 60 minutes time slot, the probability scores on a minute-by-minute basis are averaged out to compute probability score for a 60 minutes time slot.

At 306, the one or more time slots (e.g., of 60, 90, 120, 180 minutes) for specific number of days whose computed probability score is greater than or equal to a pre-determined threshold probability score may be determined. Each of the time slots per day correspond to the virtual operating room associated with the hospital. The pre-determined threshold probability score may have a value ranging from 0.0 to 1.0. The pre-determined threshold probability score is computed to maximize recall and precision with a strong preference for higher precision which means reducing recall. The higher precision associated with the pre-determined threshold probability may minimize cancellation of scheduled medical procedures due to unavailability of operating rooms in a hospital. The pre-determined threshold probability score may vary from one hospital to another. Also, the pre-determined threshold probability score may be adjusted from time to time in order to ensure lesser recall and higher precision at all times. The higher the pre-determined threshold probability score, the higher the density of true positives will be (i.e., guarantee of availability of time slots in one of the physical operating rooms). The probability scores equal to or greater than the pre-determined threshold probability score may indicate a higher chance of time slot available on a specific day for performing a medical procedure even when all physical operating rooms in a hospital are shown as completely booked.

At 308, the available time slots for each day of specific number of days may be displayed in the virtual operating room associated with the hospital on a graphical user interface. These time slots indicate that additional requests for medical procedures from doctors can be accommodated in a particular hospital for a specific number of days.

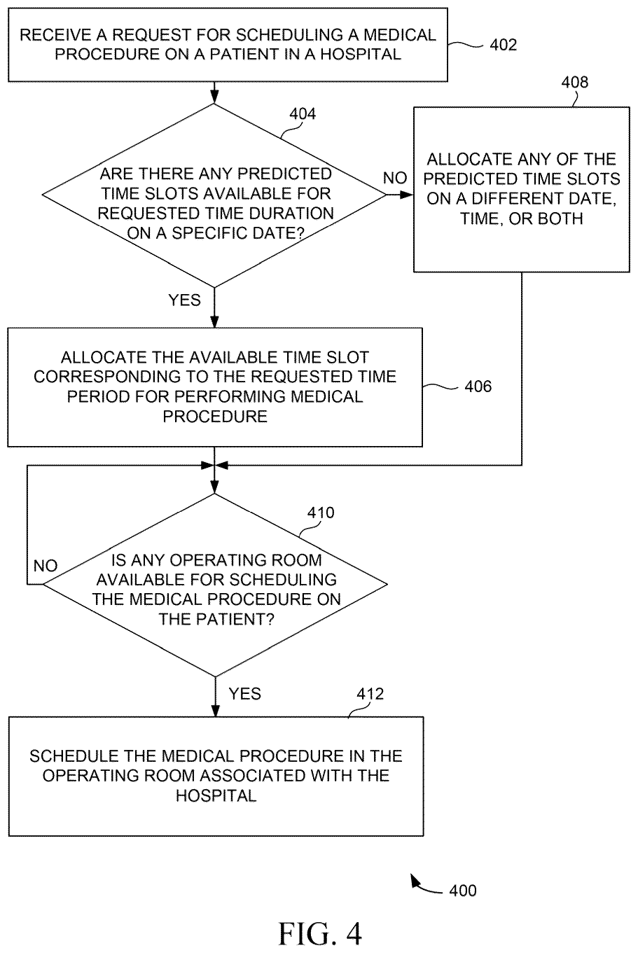

FIG. 4 is a flow diagram 400 illustrating an example method of scheduling a medical procedure on a patient in a hospital based on a request for scheduling the medical procedure. At 402, a request for scheduling a medical procedure on a patient in a hospital may be received. In an example, the request may include a specific date and a time duration required for performing the medical procedure on the patient. The request may also include procedure identifier, surgeon name, patient type, and/or patient identifier.

At 404, it is determined whether any of predicted time slots are available for the requested time duration on the specific date. In an example, as described in FIG. 3, availability of time slots in a hospital for performing medical procedures is predicted using a trained artificial intelligence model. In this example, method 400 may determine whether any of the predicted time slots is available as per the request received at 402.

If the predicted time slot for the requested time duration on the specific date is available, at 406, the predicted time slot may be allocated in the virtual operating room for performing the medical procedure on the patient. If the predicted time slot for the requested time duration is not available, at 408, an available time slot from the predicted time slots may be recommended to a doctor (e.g., via an EMR system). The available predicted time slot may fall on a different date/time than the specific date/time. If the doctor agrees to one of the recommended time slots, the available time slot is allocated for performing medical procedure on the patient. The virtual operating room is updated with information associated with the allocated time slot.

At 410, it is determined whether any operating room associated with the hospital has become available for scheduling the medical procedure on the patient. For example, it is determined from an EMR system of a hospital whether any operating room has become available for the time duration required for performing the medical procedure. If the operating room has become available, at 412, the medical procedure may be scheduled in the available operating room associated with the hospital In an example, the medical procedure is scheduled in the available operating room as per the allocated time slot. In another example, the medical procedure is scheduled in the available operating room as per time slot available in the EMR system. In some examples, the doctor associated with the medical procedure is notified that the medical procedure is scheduled in the operating room for a different time slot. If the doctor accepts the time slot, then the EMR system of the hospital is updated with the details of the medical procedure and the operating room. Additionally, the virtual operating room is also updated after the medical procedure is scheduled in the operating room. In this manner, additional requests for medical procedures may be accommodated in a hospital using a trained artificial intelligence model.

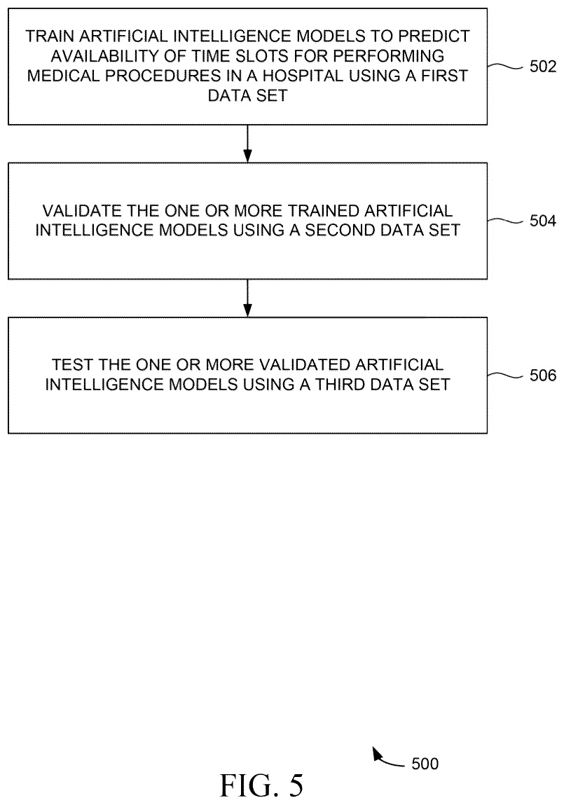

FIG. 5 is a flow diagram 500 illustrating an example method of training artificial intelligence models to predict availability of time slots for performing medical procedures in hospitals. At 502, artificial intelligence models may be trained to predict availability of time slots for performing medical procedures in a hospital using a first data set. In an example, artificial intelligence models may be binary supervised classification models such as linear regression model, support vector machine model, random forest model and the like. At 504, the one or more trained artificial intelligence models may be validated using a second data set. At 506, the one or more validated artificial intelligence models may be tested using a third data set.

For example, a data set is generated for training, testing, and validating artificial intelligence models. In one example, raw operating room scheduling data is transformed into feature vectors which are stored in a BigQuery table. A ground truth on usage of operating rooms in a hospital is computed on minute-by-minute basis. For example, each day may be divided into 480 minutes on a “prime day” The ground truth for each minute may be calculated as a binary value. The ground truth for a specific minute with value ‘TRUE’ may indicate whether at least one physical operating room is available for a specific period following the specific minute. The features extracted from historical operating room schedule data and real-time operative room schedule data may include operating room usage patterns throughout the day, unique operating schedule repeating patterns of doctors and allocated time slots, scheduling variability due to week of a month, number of operating rooms scheduled at a given time, average operating rooms scheduled prior at a given time, specific operating rooms scheduled, cancelled medical procedures, and the like. Historical operating room scheduling data may also include historical recorded elements which have a predictive value in terms of being possible determinants of variance from an operating room schedule. Historical recorded elements may include actual in-room time, incision time, incision close time, actual out time, post-anaesthesia and recovery time. Data sets are generated using the ground truth and the feature vectors.

The data set is divided into a first data set, a second data set, and a third data set. A first data set is the largest data set (e.g., about 80% of the data set) used to train artificial intelligence models. A second data set (e.g., about 10%) is used to observe the impact of tuning various algorithmic parameters and to prevent the trained artificial intelligence models from being too closely optimized for the first data set. The third data set is used to provide an authoritative assessment of performance of the validated artificial intelligence models. In some examples, artificial intelligence models are trained, validated, and tested using the first data set, the second data set, and the third data set corresponding to entire days. For testing, the third data set may include most recent data (e.g., real-time operating room scheduling data) to better match real-world expectations for scheduling medical procedures in future.

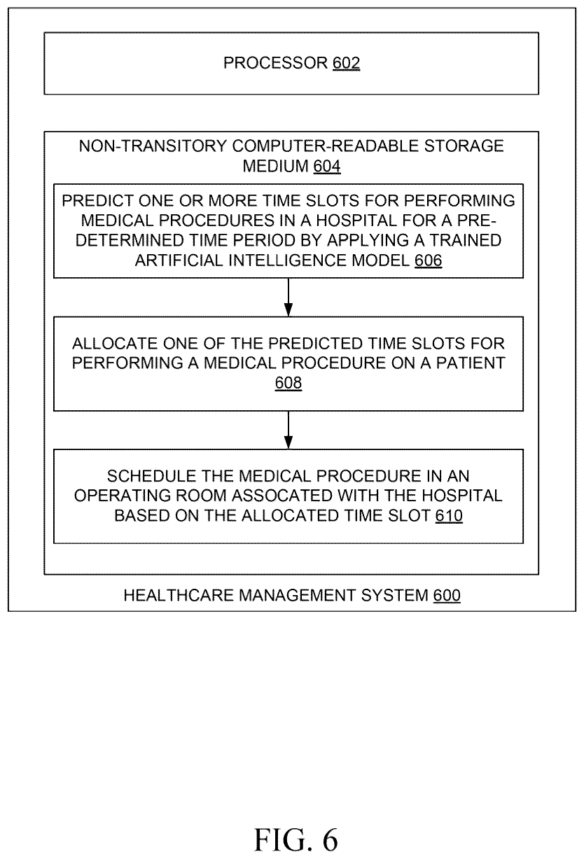

FIG. 6 is a block diagram of an example healthcare management system 600 including non-transitory computer-readable storage medium 604 storing instructions to schedule a medical procedure in an operating room of a hospital using a trained artificial intelligence model. Healthcare management system 600 may include processor 602 and computer-readable storage medium 604 communicatively coupled through a system bus. Processor 602 may be any type of central processing unit (CPU), microprocessor, or processing logic that interprets and executes computer-readable instructions stored in computer-readable storage medium 604. Computer-readable storage medium 604 may be a random-access memory (RAM) or another type of dynamic storage device that may store information and computer-readable instructions that may be executed by processor 602. For example, computer-readable storage medium 604 may be synchronous DRAM (SDRAM), double data rate (DDR), Rambus® DRAM (RDRAM), Rambus® RAM, etc., or storage memory media such as a floppy disk, a hard disk, a CD-ROM, a DVD, a pen drive, and the like. In an example, computer-readable storage medium 604 may be a non-transitory computer-readable storage medium. In an example, computer-readable storage medium 604 may be remote but accessible to healthcare management system 600.

Computer-readable storage medium 604 may store instructions 606, 608, and 610. Instructions 606 may be executed by processor 602 to predict one or more time slots for performing medical procedures in hospital for a pre-determined time period by applying a trained intelligence learning model on real-time operating room scheduling data of the hospital. In some examples, instructions 606 to predict the time slots for performing the medical procedures may include instructions to:
create a virtual operating room associated with the hospital, and
predict one or more time slots for performing the medical procedures in the hospital for the pre-determined time period by applying the trained artificial intelligence model on the real-time operating room scheduling data of the hospital.

Each of the time slots may correspond to different days in the virtual operating room. In some examples, instructions may be executed by processor 602 to display the one or more time slots in the virtual operating room associated with the hospital on a graphical user interface (e.g., of an EMR system).

In other examples, instructions 606 to predict availability of the time slots for the pre-determined time period may include instructions to:
compute a probability score indicating availability of each of the time slots for the pre-determined time period by applying the trained artificial intelligence model on the real-time operating room scheduling data of the hospital, and
determine the one or more time slots whose computed probability score is greater than or equal to pre-determined threshold probability score.

In yet another examples, instructions 606 to predict availability of the time slots for performing the medical procedures may include instructions to predict availability of the time slots for performing the medical procedures for the pre-determined time period by applying the trained artificial intelligence model on the real-time operating room scheduling data in response to an electronic medical record system of the hospital indicating non-availability of the operating rooms in the hospital for the pre-determined time period.

Instructions 608 may be executed by processor 602 to allocate one of the predicted time slots for performing a medical procedure on a patient in the hospital in response to a request for scheduling the medical procedure on the patient. In an example, instructions 608 to allocate the time slot for performing the medical procedure on the patient may include instructions to allocate the time slot corresponding to the virtual operating room for performing the medical procedure on the patient. In other examples, instructions 608 to allocate the time slot for performing the medical procedure on the patient may include instructions to:
receive a request for scheduling the medical procedure on the patient in the hospital, wherein the request comprises a specific date and a time duration required for performing the medical procedure,
determine whether any of the predicted time slots are available for the requested time duration on the specific date,
allocate the available time slot for performing the medical procedure if the time slot on the specific date is available, and
allocate any of the predicted time slots for the requested time duration if the time slot on the specific date is not available.

Instructions 610 may be executed by processor 602 to schedule the medical procedure in one of a plurality of operating rooms associated with the hospital based on the allocated time slot when the operating room becomes available for performing the medical procedure. In an example, instructions 610 to schedule the medical procedure in one of the plurality of operating rooms associated with the hospital may include instructions to:
determine whether any of the plurality of operating rooms in the hospital is available for performing the medical procedure using the real-time operating room scheduling data associated with the hospital,
schedule the medical procedure in one of a plurality of operating rooms in the hospital if said one of the plurality of operating rooms in the hospital is available, and
repeat determining whether any of the plurality of operating rooms in the hospital is available for performing medical procedure till the arrival of allocated time slot for performing of the medical procedure.

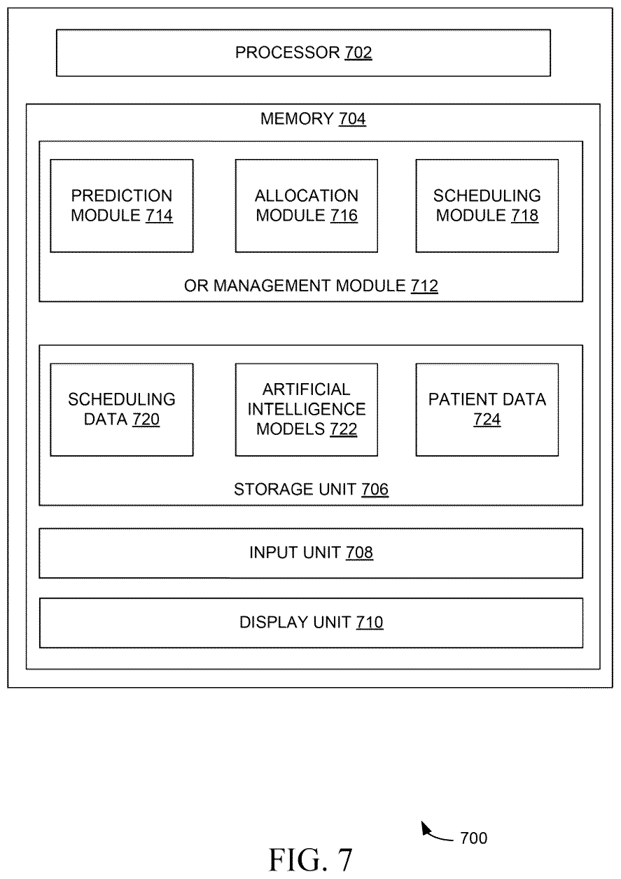

FIG. 7 is a block diagram of an example healthcare management system 700 for scheduling a medical procedure in an operating room of a hospital. Example healthcare management system 700 may be a personal computer, workstation, laptop computer, tablet computer, and the like. In FIG. 7, healthcare management system 700 includes a processor 702, a memory 704, a storage unit 706, an input unit 708, and a display unit 710.

Processor 702, as used herein, may be any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. Processor 702 may also include embedded controllers, such as generic or programmable logic devices or arrays, application specific integrated circuits, single-chip computers, and the like.

Memory 704 may be non-transitory volatile memory and non-volatile memory. Memory 704 may be coupled for communication with processor 702, such as being a computer-readable storage medium. Processor 702 may execute instructions and/or code stored in memory 704. A variety of computer-readable instructions may be stored in and accessed from memory 704. Memory 704 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like.

In an example, memory 704 in ludes an OR management module 712 stored in, the form of machine-readable instructions on any of the above-mentioned storage media and may be in communication to and executed by processor 702. OR management module 712 includes a prediction module 714, an allocation module 716, and a scheduling module 718. Prediction module 714 may predict one or more available time slots for performing medical procedures in a hospital using one or more trained artificial intelligence models. Allocation module 716 may allocate one of the available time slots for performing a medical procedure on a patient in the hospital in response to a request to schedule the medical procedure on the patient, Scheduling module 718 may schedule the medical procedure on the patient in one of a plurality of operating rooms in the hospital based on the allocated time slot when the operating room becomes available for scheduling the medical procedure.

Storage unit 706 may be a non-transitory storage medium that stores scheduling data 720, artificial intelligence models 722, and patient data 724. Scheduling data 720 may include real-time scheduling data associated with hospitals. For example, the real-time scheduling data is received from EMRs associated with hospitals. Real-time scheduling data includes the schedule of medical procedures in operating rooms of each hospital. Real-time scheduling data is updated regularly at short intervals in storage unit 706 from the respective EMRs. Artificial intelligence models 722 may include trained artificial intelligence models for predicting one or more available time slots for performing medical procedures in each hospital based on real-time scheduling data. Patient data 724 may include data associated with patients on which medical procedure is to be performed. Patient data 724 may include patient identifier, patient type, procedure identifier, doctor's name, and the like. Patient data 724 may be used for allocating time slot for performing a medical procedure and fir scheduling the medical procedure in an operating room of a hospital.

Input unit 708 may include input devices such as keypad, touch-sensitive display, camera (e.g., a camera receiving gesture based inputs), and the like capable of receiving input signals such as a request for scheduling a medical procedure in an operating room of a hospital from a doctor's office. Display unit 710 may be a device with a graphical user interface displaying predicted time slots corresponding to a virtual operating room for a specific umber of days.

Those having ordinary skill in the art will appreciate that the hardware depicted in FIG. 7 may vary for particular implementations. For example, other peripheral devices such as an optical disk drive and the like, Local Area Network (LAN)/Wide Area Network (WAN)/Wireless (e.g., Wi-Fi) adapter, graphics adapter, disk controller, input/output (I/O) adapter also may be used in addition to or in place of the hardware depicted. The depicted example is provided for the purpose of explanation only and is not meant to imply architectural limitations with respect to the present disclosure.

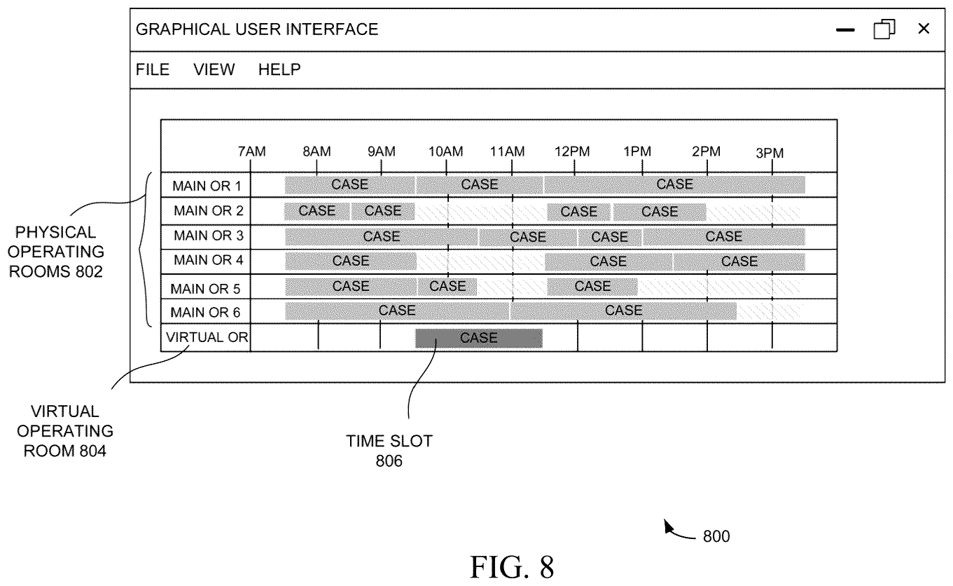

FIG. 8 is a diagrammatic representation of a graphical user interface 800 displaying predicted availability of a time slot 806 in a virtual operating room 804. Example graphical user interface 800 may be a component of an electronic medical record (EMR) system. Graphical user interface 800 shows schedule of physical operating rooms 802 for a specific day. Also, graphical user interface 800 shows schedule of a virtual operating room 804 for the same day. Virtual operating room 804 may indicate that time slot 806 is predicted to be available for performing a medical procedure. Thus, when a request for scheduling a medical procedure is received by an OR management module (e.g., OR management module 116 of FIG. 1), the OR management module may check whether a requesting doctor has block time in any of the physical operating rooms 802. If the requesting doctor has block time in any of the physical operating rooms 802, then the OR management module may schedule the medical procedure into a physical operating room 802 where the doctor has block time.

If the doctor does not have block time, then the OR management module may check whether there are any free/open time slots in any of the physical operating rooms 802. If there are free/open time slots in any of the physical operating rooms 802, then the OR management module may schedule the medical procedure in an available physical operating room 802. If none of the physical operating rooms 802 are available, then OR management module may allocate time slot 806 in the virtual operating room to the doctor. Time slot 806 indicates a good chance of one of the physical operating rooms 802 becoming available on a specific date. The OR management module may schedule the medical procedure in a physical operating room 802 of the hospital as soon as the physical operating room 802 becomes available prior to the date of the medical procedure. In the meantime, any tests and/or processes related to the medical procedure are carried out once the time slot 806 in the virtual operating room is allocated. In this manner, optimized utilization of physical operating rooms in a hospital by predicting available time slots for performing medical procedures using trained artificial intelligence model may be achieved.

The above-described examples are for the purpose of illustration. Although the above examples have been described in conjunction with example implementations thereof, numerous modifications may be possible without materially departing from the teachings of the subject matter described herein. Other substitutions, modifications, and changes may be made without departing from the spirit of the subject matter. Also, the features disclosed in this specification (including any accompanying claims, abstract, and drawings), and any method or process so disclosed, may be combined in any combination, except combinations where some of such features are mutually exclusive.

The terms “include,” “have,” and variations thereof, as used herein, have the same meaning as the term “comprise” or appropriate variation thereof. Furthermore, the term “based on”, as used herein, means “based at least in part on.” Thus, a feature that is described as based on some stimulus can be based on the stimulus or a combination of stimuli including the stimulus. In addition, the terms “first” and “second” are used to identify individual elements and may not meant to designate an order or number of those elements.

The present description has been shown and described with reference to the foregoing examples. It is understood, however, that other forms, details, and examples can be made without departing from the spirit and scope of the present subject matter that is defined in the following claims.

What is claimed is:

1. A method of scheduling a medical procedure associated with a patient in an operating room associated with a hospital, comprising:
predicting availability of one or more time slots for performing medical procedures in a hospital for a pre-determined time period by applying a trained artificial intelligence model on real-time operating room scheduling data associated with the hospital;
in response to a request for scheduling a medical procedure on a patient, allocating one of the predicted time slots for performing the medical procedure in the hospital; and
scheduling the medical procedure in one of a plurality of operating rooms associated with the hospital based on the allocated time slot when the operating room becomes available for performing the medical procedure.

2. The method of claim 1, wherein predicting the one or more time slots for performing the medical procedures in the hospital for the pre-determined time period comprises:
creating a virtual operating room corresponding to the plurality of operating rooms associated with the hospital; and
predicting the one or more time slots for performing the medical procedures in the hospital for the pre-determined time period by applying the trained artificial intelligence model on the real-time operating room scheduling data associated with the hospital, wherein each of the time slots corresponds to the virtual operating room.

3. The method of claim 2, further comprising:
displaying the one or more time slots in the virtual operating room associated with the hospital on a graphical user interface.

4. The method of claim 2, wherein allocating the time slot for performing the medical procedure on the patient comprises:
allocating the time slot corresponding to the virtual operating room for performing the medical procedure on the patient.

5. The method of claim 4, wherein predicting availability of the one or more time slots for the pre-determined time period comprises:
computing a probability score indicating availability of each of the time slots for the pre-determined time period by applying the trained artificial intelligence model on the real-time operating room scheduling data of the hospital; and
determining the one or more time slots whose computed probability score is greater than or equal to pre-determined threshold probability score.

6. The method of claim 1, wherein predicting availability of the time slots for performing the medical procedures in the hospital using the trained artificial intelligence model comprises:
in response to an electronic medical record system of the hospital indicating non-availability of the operating rooms in the hospital for the pre-determined time period, predicting availability of the one or more time slots for performing the medical procedures for the pre-determined time period by applying the trained artificial intelligence model on the real-time operating room scheduling data associated with the hospital.

7. The method of claim 1, wherein scheduling the medical procedure in the one of the plurality of operating rooms associated with the hospital comprises:
determining whether any of the plurality of operating rooms in the hospital is available for performing the medical procedure using the real-time operating room scheduling data associated with the hospital; and
if one of the plurality of operating rooms in the hospital is available, scheduling the medical procedure in said one of a plurality of operating rooms in the hospital.

8. The method of claim 1, wherein allocating the time slot for performing the medical procedure on the patient comprises:
receiving a request for scheduling the medical procedure on the patient in the hospital, wherein the request comprises a specific date and a time duration required for performing the medical procedure;
determining whether any of the predicted time slots are available for the requested time duration on the specific date;
if the time slot on the specific date is available, allocating the available time slot for performing the medical procedure for the requested time duration; and
if the time slot on the specific date is not available, allocating any of the predicted time slots for the requested time duration.

9. The method of claim 1, further comprising:
training one or more artificial intelligence models to predict availability of the one or more time slots for performing the medical procedures in the hospital using a first data set, wherein each of the one or more artificial intelligence models is trained to predict availability of the one or more time slots of specific time duration;
validating the one or more trained artificial intelligence models using a second data set; and
testing the one or more validated artificial intelligence models using a third data set.

10. The method of claim 9, wherein the first data set, the second data set, and the third data set are computed using historical operating room scheduling data associated with the hospital and the real-time operating room scheduling data associated with the hospital.

11. A system for scheduling a medical procedure associated with a patient in an operating room associated with a hospital, comprising:
a processor; and
memory coupled to the processing unit, wherein the memory comprises an OR management module operable to:
predict one or more time slots for performing medical procedures in a hospital for a pre-determined time period by applying a trained artificial intelligence model on real-time operating room scheduling data associated with the hospital;
in response to a request for scheduling a medical procedure on a patient, allocate one of the predicted time slots for performing the medical procedure in the hospital; and
schedule the medical procedure in one of a plurality of operating rooms associated with the hospital based on the allocated time slot when the operating room becomes available for performing the medical procedure.

12. The system of claim 11, wherein the OR management module is to:
create a virtual operating room associated with the hospital; and
predict the one or more time slots for performing the medical procedures in the hospital for the pre-determined time period by applying the trained artificial intelligence model on the real-time operating room scheduling data associated with the hospital, wherein each of the time slots correspond to the virtual operating room.

13. The system of claim 12, wherein the OR management module is to display the one or more time slots in the virtual operating room associated with the hospital on a graphical user interface.

14. The system of claim 12, wherein the OR management module is to allocate the time slot corresponding to the virtual operating room for performing the medical procedure on the patient.

15. The system of claim 14, wherein the OR management module is to:
compute a probability score indicating availability of each of the time slots for the pre-determined time period by applying the trained artificial intelligence model on the real-time operating room scheduling data of the hospital; and
determine the one or more time slots whose computed probability score is greater than or equal to pre-determined threshold probability score.

16. The system of claim 12, wherein the OR management module is to:
predict availability of the one or more time slots for performing the medical procedures for the pre-determined time period by applying the trained artificial intelligence model on the real-time operating room scheduling data associated with the hospital in response to an electronic medical record system of the hospital indicating non-availability of the operating rooms in the hospital for the pre-determined time period.

17. The system of claim 11, wherein the OR management module is to:
determine whether any of the plurality of operating rooms in the hospital is available for performing the medical procedure using the real-time operating room scheduling data associated with the hospital; and
if one of the plurality of operating rooms in the hospital is available, schedule the medical procedure in said one of a plurality of operating rooms in the hospital.

18. The system of claim 11, wherein the OR management module is to:
receive a request for scheduling the medical procedure on the patient in the hospital, wherein the request comprises a specific date and a time duration required for performing the medical procedure;
determine whether any of the predicted time slots are available for the requested time duration on the specific date;
allocate the available time slot for performing the medical procedure for the requested time duration if the time slot on the specific date is available; and
allocate any of the predicted time slot for the requested time duration if the time slot on the specific date is not available.

19. A non-transitory computer-readable storage medium storing instructions executable by a processing unit of a system to:
predict, by an OR management module, one or more time slots for performing medical procedures in a hospital for a pre-determined time period by applying a trained artificial intelligence model on real-time operating room scheduling data associated with the hospital;
in response to a request for scheduling a medical procedure on a patient, allocate one of the predicted time slots for performing the medical procedure in the hospital; and
schedule the medical procedure in one of a plurality of operating rooms associated with the hospital based on the allocated time slot when the operating room becomes available for performing the medical procedure.

20. The non-transitory computer-readable storage medium of claim 19, wherein the instructions to predict the one or more time slots for performing the medical procedures in the hospital for the pre-determined time period comprises instructions to:
create a virtual operating room associated with the hospital; and
predict the one or more time slots for performing the medical procedures in the hospital for the pre-determined time period by applying the trained artificial intelligence model on the real-time operating room scheduling data associated with the hospital, wherein each of the time slots correspond to the virtual operating room.

21. The non-transitory computer-readable storage medium of claim 19, further comprising instructions to display the one or more time slots in the virtual operating room associated with the hospital on a graphical user interface.

22. The non-transitory computer-readable storage medium of claim 20, wherein the instructions to allocate the time slot for performing the medical procedure on the patient comprises instructions to allocate the time slot corresponding to the virtual operating room for performing the medical procedure on the patient.

23. The non-transitory computer-readable storage medium of claim 22, wherein the instructions to predict availability of the one or more time slots for the pre-determined time period comprises instructions to:
compute a probability score indicating availability of each of the time slots for the pre-determined time period by applying the trained artificial intelligence model on the real-time operating room scheduling data of the hospital; and
determine the one or more time slots whose computed probability score is greater than or equal to pre-determined threshold probability score.

24. The non-transitory computer-readable storage medium of claim 19, wherein the instructions to predict availability of the time slots for performing the medical procedures in the hospital comprise instructions to:
predict availability of the one or more time slots for performing the medical procedures for the pre-determined time period by applying the trained artificial intelligence model on the real-time operating room scheduling data associated with the hospital in response to an electronic medical record system of the hospital indicating non-availability of the operating rooms in the hospital for the pre-determined time period.

25. The non-transitory computer-readable storage medium of claim 19, wherein the instructions to schedule the medical procedure in said one of the plurality of operating rooms associated with the hospital instructions to:
determine whether any of the plurality of operating rooms in the hospital is available for performing the medical procedure using the real-time operating room scheduling data associated with the hospital; and
if one of the plurality of operating rooms in the hospital is available, schedule the medical procedure in said one of the plurality of operating rooms in the hospital.

26. The non-transitory computer-readable storage medium of claim 19, wherein the instructions to allocate one of the one or more time slots for performing the medical procedure on the patient comprises instructions to:
receive a request for scheduling the medical procedure on the patient in the hospital, wherein the request comprises a specific date and a time duration required for performing the medical procedure;
determine whether any of the predicted time slots are available for the requested time duration on the specific date;
allocate the available time slot for performing the medical procedure for the requested time duration if the time slot on the specific date is available; and
allocate any of the predicted time slot for the requested time duration if the time slot on the specific date is not available.
